(19) United States
(12) Patent Application Publication
(10) Pub. No.: US 2009/0012352 A1
(43) Pub. Date: Jan. 8, 2009

(54) RECOVERABLE INTRA-UTERINE DEVICE

(75) Inventors: Pascal Mock, Geneve (CH); Nicolas Bouche, Les Paccots (CH); Philippe Le Goff, Epalinges (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Anecova SA, Suisse (CH)

(21) Appl. No.: 12/159,374

(22) PCT Filed: Dec. 26, 2006
(86) PCT No.: PCT/IB2006/004136

§ 371 Date: Jun. 26, 2008

(30) Foreign Application Priority Data
Dec. 26, 2005 (FR) 05/13317

(51) Int. Cl. A61B 17/435

(52) U.S. Cl. 600/34

(57) ABSTRACT

A recoverable intra-uterine device comprising a housing containing one or several elements selected from a group containing an embryo, male and/or female gametes, a fertilised oocyte, an unfertilised egg and the combination thereof, wherein said housing is provided with a wall made of a biocompatible material. The wall is provided with a series of perforations whose size is sufficient in order to bring the intra-uterine medium into a cellular contact with the housing and to keep the elements therein. The inventive device is provided with a system for loading and unloading one or several elements selected from the group containing an embryo, male and/or female gametes, a fertilised oocyte, an unfertilised egg and the combination thereof.

The present invention concerns a recoverable intra-uterine device, used in particular to implant gametes or embryos in the uterus.

The present invention is generally concerned with the field of in vivo and in utero fertilization as used in assisted reproduction methods utilizing fertilization and/or preimplantation development techniques.

A recoverable intra-uterine device of this kind known in the art is described in the document WO03/011200.

The intra-uterine device includes a housing adapted to contain the elements to be encapsulated in the device, for example an embryo, male and/or female gametes, a fertilized ovocyte, or an unfertilized egg.

This encapsulation technique is used for the fertilization and development of an embryo inside a capsule placed in the uterus.

It has the advantage of overcoming a certain number of problems linked to in vitro culture. Thus by placing the capsule with a developing embryo temporarily in the uterus, the gametes and/or the embryos are placed in a natural environment during preimplantation development, before final transfer of the embryo into the uterus.

This enables complex interactions between the endometrium of the uterus and the gametes and/or the embryos.

The Applicant has established that embryos obtained in this way are of better quality not only from the morphological point of view but also in terms of their capacity to develop and to be re-implanted in the uterus subsequently.

Furthermore, by minimizing the interaction of the embryo with a culture medium, the risks of the embryo being degraded by foreign factors are reduced.

By obtaining embryos of better quality, it is normally possible to reduce occurrences of multiple pregnancies by allowing the re-implantation of a single embryo with much greater chances of success than the standard methods of assisted reproduction.

Moreover, because fertilization and preimplantation development occur directly in the uterus, the psychological impact on the couple can be considerable as the assisted reproduction process is closer to natural conception and requires greater involvement of the couple.

The recoverable intra-uterine device described in the document WO03/011200 includes a housing the wall of which is made from a permeable porous membrane.

The choice of a porous membrane of this kind was initially intended to protect gametes and/or embryos placed in the device whilst enabling nutrients present in the uterine medium to pass into the interior of the housing of the device.

However, a permeable porous membrane is fragile and handling of this capsule, in particular when loading and unloading encapsulated elements, or when implanting the capsule in or recovering it from the uterus, could damage the wall of the capsule and possibly cause the loss of the encapsulated elements.

Moreover, the production of a device using this kind of porous membrane to guarantee optimum reproducibility of the implants is difficult and complex.

Furthermore, exchanges between the interior of the housing and the uterine matrix are limited to elements of small size, such as oxygen proteins or other hormones, the size of which is less than one million Dalton.

However, the Applicant has found that exchanges between the elements contained in the capsule, in particular the embryos, and elements of greater size, in particular cells, contained in the uterine fluid, could further enhance the development of the embryos in the capsule.

An object of the present invention is to eliminate the drawbacks of the porous membranes described hereinabove and to propose an intra-uterine device that enhances exchanges with the uterine matrix in a reliable and reproducible manner.

Thus, according to the invention, the wall of the housing includes a series of perforations of sufficient size to allow cellular contact between the uterine matrix and the housing.

Thus, by producing perforations in the membrane, it is possible reliably and reproducibly to produce openings enabling cells to pass between the interior and the exterior of the device.

The reproduction inside the housing of a medium close to that present in the uterine matrix, which comprises uterine fluids coming both from the uterine cavity and from the fallopian tubes, could favor the development of the embryos. Furthermore, exchange of cells could probably facilitate subsequent re-implantation and nidation of the embryo in the uterus.

Moreover, producing perforations in the wall of the housing allows the use of materials that are non-porous or with which it would be difficult to produce a porous structure.

It is therefore possible by controlling the size of the perforations and the nature of the material used to produce a device having a wall that is sufficiently permeable to enable cellular interaction with the cells of uterine matrix whilst having sufficient rigidity to make manipulation of the device, in particular with a view to loading embryos or gametes and introducing the device into the uterus and removing it therefrom, safe.

The device according to the invention can therefore be loaded and unloaded without degrading the quality of the embryos or other elements loaded in the housing.

The phenomena of interaction with the uterine medium, enhanced compared to the state of the art, could probably be of a kind to favor the development of the encapsulated elements, and in particular the development of the embryo or the fertilization of the implanted eggs.

In practice, the perforations have dimensions between 10 and 150 μm.

Creating perforations having an aperture of at least 10 μm makes it possible for cells with a size between 5 and 10 μm to pass into the interior of the housing.

Moreover, the perforations have an aperture at most equal to 150 μm in their largest dimension.

This maximum aperture of the perforations enables the wall of the housing to exercise its boundary function to the full, allowing exchanges with the uterine matrix whilst retaining the encapsulated elements, such as in particular ovocytes and/or embryos that have a minimum dimension of 150 μm, inside the housing.

In one embodiment, the perforations are uniformly distributed in the wall of the housing and disposed in a quincunx formation.

It is therefore possible to favor cellular interaction uniformly.

In one embodiment of the invention, the wall is produced in a biocompatible transparent material.

The use of a transparent material facilitates handling the device, in particular when loading and unloading the device, thanks to seeing the gametes or embryos in the device.

It is furthermore possible to observe the development of the ovocytes and/or embryos encapsulated inside the device from the exterior of the device.

In practice, the wall can be produced in polymer, elastomer, of silicone or polyurethane type, or in ceramic, in glass, or in a metal alloy.

A second aspect of the invention concerns the use of the inter-uterine device for loading and/or unloading elements chosen from the group comprising an embryo, male and/or female gametes, a fertilized ovocyte, an unfertilized egg and a combination of those elements. This use of the device enables elements to be loaded into and unloaded from the housing without degrading them.

Other features and advantages of the invention will become more apparent in the course of the following description.

In the appended drawings, provided by way of nonlimiting example:

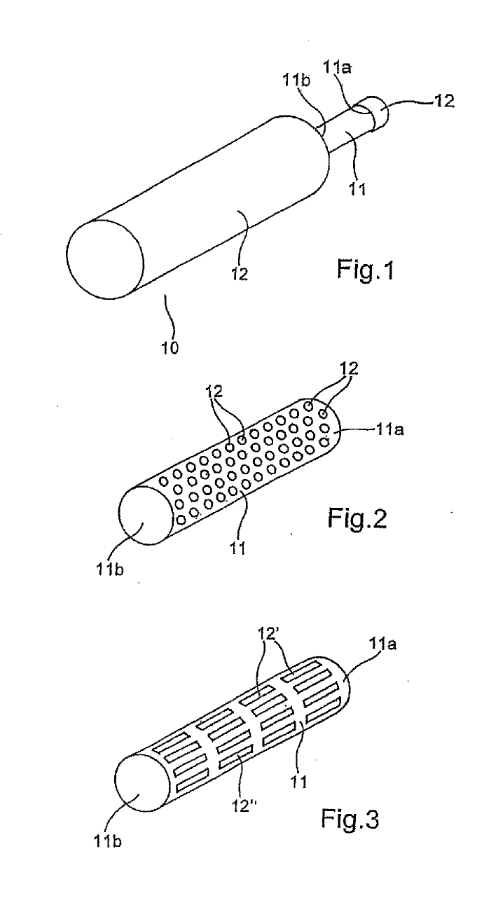

FIG. 1 is a perspective view of one embodiment of an intra-uterine device of the invention;

FIG. 2 is a diagrammatic perspective view of the housing of the FIG. 1 intra-uterine device; and

FIG. 3 is a diagrammatic perspective view to a larger scale of the housing of the FIG. 1 intra-uterine device according to a second embodiment of the invention.

The general structure of an intra-uterine device according to the invention is described first with reference to FIG. 1.

The intra-uterine device 10 shown in FIG. 1 is used in assisted fertilization techniques to implant and temporarily maintain male and female gametes (in vivo fertilization) and/or embryos (preimplantation development) in a uterine cavity.

In principle, in an in vitro assisted fertilization method, this intra-uterine device enables one or more elements to be loaded into a permeable housing 11 at the start of embryo development, the device to be introduced into the uterine cavity for a predetermined period (from a few hours to a few days), and the intra-uterine device thereafter to be recovered to extract the embryo or embryos in order to implant them in the uterine cavity.

The housing 11 can be open at a distal end 11a to allow the loading of elements (embryos, male and/or female gametes, fertilized ovocytes, unfertilized eggs) into the housing 11. A stopper 12 is adapted to close off this distal end 11a.

The housing 11 is moreover fixed at its proximal end 11b to a silicone element 12 that can itself be connected to an element for holding the capsule, made of stainless steel, for example.

This device 10 furthermore cooperates with accessories for placing the device in the uterine cavity.

Those accessories are not described in more detail here, as they are not directly related to the present invention. The document WO03/011200 can advantageously be consulted for technical information relating to the use of the intra-uterine device of the invention.

In this embodiment the housing 11 has a substantially cylindrical shape. The length of the housing can be between 1 and 25 mm, and preferably equal to about 10 mm. The outside diameter of this housing is between 0.5 and 2 mm, and preferably substantially equal to 0.9 mm. The inside diameter of the housing is between 0.4 and 1.8 mm, and preferably equal to 0.8 mm.

The membrane of the housing 11 therefore has a thickness of the order of 0.1 mm.

The wall can be produced in different materials.

In particular, this wall can be produced in a porous polymer, of the polyethersulfone (PES), polyacrylate, acrylate copolymer or polyvinylidiene type.

It can also be produced in a plastic material of the silicone or polyurethane type, or in ceramic or in glass.

It can also be produced in a metal alloy of the stainless steel type or titanium or titanium alloy.

Generally speaking, the materials used must be tested to be non-toxic, biocompatible and stable in use.

The configuration and the size of the housing 11 enable it to be placed in the uterine cavity using a transfer catheter having an inside diameter of 1 cm. A catheter of this kind is adapted to pass through the cervical canal, which generally has a diameter of about 1.5 cm. The ovocytes, surrounded by complex cells, developing in the housing have a size between 350 and 400 μm, and so a housing having an inside diameter of 0.8 mm is sufficient to contain between 5 and 10 embryos.

It the wall of the housing 11 is a porous membrane of the PES type, this allows nutrients contained in the uterine fluid to pass into the interior of the housing to come into contact with the developing embryo.

However, to enhance permeability and allow cellular contact between the interior of the housing 11 and the intra-uterine medium, the housing 11 is provided with perforations 12.

FIG. 2 shows a first embodiment of a perforated wall.

These perforations can be produced in the wall by laser cutting, for example.

Other techniques of producing perforations could be used, of course.

Using laser techniques makes it possible to obtain perforations having a minimum dimension of 10 μm.

Moreover, these perforations 12 have a maximum dimension of 150 μm, so as to retain in the interior of the housing ovocytes and/or embryos whose dimensions are of the order of 150 μm.

In the FIG. 2 embodiment, the perforations 12 are of circular shape, and preferably have a diameter between 40 and 75 μm. That diameter can be equal to 50 μm, although this is not limiting on the invention.

In this embodiment, in which the housing is of elongate cylindrical shape, the wall comprises at least one, and in this embodiment several, series of perforations 12 aligned in the longitudinal direction of the housing 11. These series of perforations are disposed parallel to each other and the perforations 12 are disposed in a quincunx formation.

The wall of the housing 11 preferably includes perforations 12 distributed uniformly over the whole of the cylindrical wall so that cellular contact with the intra-uterine medium can be obtained all around the housing 11.

The distances between the perforations are between 20 and 200 μm, and so the wall of the housing 11 is highly permeable.

This embodiment is in no way limiting on the invention, of course, in particular in terms of the distribution, shape and size of the perforations.

For example, FIG. 3 shows a second embodiment of the invention in which the housing has perforations 12′ of rectangular shape.

Those perforations can have a width between 10 and 150 μm, preferably equal to 75 μm, and a length greater than or equal to 150 μm, and possibly equal to the length of the housing 11.

The housing being of elongate cylindrical shape, the rectangular perforations 12′ are aligned in the longitudinal direction of the housing.

In this embodiment, the wall includes a plurality of series of perforations 121, the series being disposed parallel to each other and uniformly distributed in the wall of the housing 11.

The distance between the perforations 12′ can also be between 20 and 200 μm.

The intra-uterine device according to the invention makes it possible to keep the ovocytes and/or the embryos in the cage-like housing and at the same time enables optimum exchange with the environment of the uterine cavity.

Of course, numerous modifications can be made to the embodiment described hereinabove without departing from the scope of the invention.

1-18. (canceled)

19. A recoverable intra-uterine device comprising a housing adapted to contain one or more elements selected from the group consisting of embryos, male and/or female gametes, fertilized ovocytes, unfertilized eggs, or combinations thereof,
the housing comprising a wall comprising biocompatible material,
wherein the wall includes a series of perforations of sufficient size to retain the elements and to allow cellular contact between the intra-uterine medium and the elements.

20. The device of claim 19, wherein the perforations have at least one dimension ranging from 10 to 150 μm.

21. The device of claim 20, wherein the perforations have at least one dimension ranging from 40 to 75 μm.

22. The device of claim 19, wherein the perforations are circular, with a diameter ranging from 40 to 75 μg.

23. The device of claim 22, wherein the diameter of the circular perforations is 50 μm.

24. The device of claim 19, wherein the perforations are rectangular, with a width ranging from 10 to 150 μm, and a length greater than 150 μm.

25. The device of claim 19, wherein the housing is of elongate cylindrical shape, and the wall comprises at least one series of perforations aligned in the longitudinal direction of the housing.

26. The device of claim 19, wherein the wall comprises a plurality of series of aligned perforations, wherein the series are disposed parallel to each other.

27. The device of claim 19, wherein the wall comprises perforations that are uniformly distributed in the wall and are disposed in at least one quincunx formation.

28. The device of claim 19, wherein the distances between the perforations range from 20 to 200 μm.

29. The device of claim 19, wherein the wall comprises a biocompatible material comprising polymer, ceramic, or glass.

30. The device of claim 19, wherein the wall comprises an elastomeric material.

31. The device of claim 19, wherein the wall comprises a transparent biocompatible material.

32. The device of claim 19, wherein the wall is comprises stainless steel, titanium, or titanium alloy.

33. The device of claim 19, wherein the perforations in the wall are made using a laser.

34. The device of claim 19, wherein the housing has a cylindrical shape with a length ranging from 1 to 25 mm, and an inside diameter ranging from 0.4 to 1.8 mm.

35. The device of claim 34, wherein the length is substantially equal to 10 mm, and the inside diameter is substantially equal to 0.8 mm.

36. The device of claim 19, wherein the device contains at least one element selected from the group consisting of male and/or female gametes, fertilized ovocytes, unfertilized eggs, or combinations thereof.

37. The device of claim 19, wherein the device contains one or more embryos.

38. A process comprising loading and/or unloading the device of claim 19 with at least one element comprising an embryo, male and/or female gametes, a fertilized ovocyte, an unfertilized egg or a combination thereof.
